(19) United States
(12) Patent Application Publication
(10) Pub. No.: US 2022/0079898 A1
(43) Pub. Date: Mar. 17, 2022

(54) SURFACTANTS FOR THE TREATMENT OF CONDITIONS THROUGH TARGETED NECROSIS

(71) Applicant: IMUNEKS FARMA ILAC SAN. VE TIC. A.S., Istanbul (TR)

(72) Inventor: Mehmet Nevzat Pisak, Istanbul (TR)

(21) Appl. No.: 17/484,494

(22) Filed: Sep. 24, 2021

Related U.S. Application Data
(63) Continuation of application No. 16/618,597, filed on Dec. 2, 2019.

(30) Foreign Application Priority Data
Jun. 2, 2017 (TR) PCT/TR2017/050242

(51) Int. Cl. A61K 31/185; A61K 9/00; A61K 9/08; A61K 31/10; A61K 47/10; A61P 31/22; A61K 9/06; A61K 47/32; A61K 47/34; A61K 47/38

(52) U.S. Cl. CPC A61K 31/185; A61K 9/0014; A61K 9/08; A61K 31/10; A61K 47/38; A61P 31/22; A61K 9/06; A61K 47/32; A61K 47/34; A61K 47/10

(57) ABSTRACT

The present invention relates to a pharmaceutical composition for use in the treatment of pre-cancerous conditions of the cervix/anogenital region, non-melanoma skin cancers (NMSCs), or actinic keratosis wherein said composition comprises a surfactant, preferably an anionic or amphoteric surfactant.

### CROSS-REFERENCE TO RELATED APPLICATIONS

This application is a continuation of U.S. patent application Ser. No. 16/618,597, filed Dec. 2, 2019, which is a national phase application under 35 U.S.C. § 371 of PCT International Application No. PCT/TR2018/050006, filed Jan. 4, 2018, which claims priority to PCT International Application No. PCT/TR2017/050242, filed Jun. 2, 2017, which are hereby incorporated by reference in its entirety.

### TECHNICAL FIELD

The present invention relates to the use of surfactants for the treatment of conditions and diseases that would benefit from the induction of necrosis and by the consequent immunomodulatory action caused by the targeted necrosis through the local application of surfactants.

### BACKGROUND ART

Programmed Cell Death (PCD) plays a fundamental role in animal development and tissue homeostasis. Abnormal regulation of this process is associated with a wide variety of human diseases, including immunological and developmental disorders, neuro-degeneration and cancer.

There are two primary forms of cell death: apoptosis and necrosis. The term apoptosis is often used interchangeably with programmed cell death. In the strictest sense, programmed cell death may be applied to other forms of cell death that require gene expression without fulfilling some, or all, of the morphological criteria of apoptosis. Apoptosis describes the collapse of a cell characterized by membrane blebbing, cell shrinkage, condensation of chromatin, and fragmentation of DNA followed by rapid engulfment of the corpse by neighbouring cells.

In an average human adult, between 50 and 70 billion cells die each day due to apoptosis simply to keep balance with the numbers of new cells arising from the body's stem cell populations. For an average child between the ages of 8 and 14, approximately 20 billion to 30 billion cells die in a day.

Necrosis on the other hand is a form of irreversible cell injury as a result of encounters with noxious stimuli which results in the premature death of cells by lysis. Such noxious stimuli include infectious agents (bacteria, viruses, fungi, parasites), oxygen deprivation or hypoxia, and extreme environmental conditions. Apoptosis can be beneficial whereas necrosis is harmful when uncontrolled.

Nonmelanoma skin cancer (NMSC) is the most common cancer in Caucasians. Basal cell carcinoma (BCC) accounts for 75% of cases, and is a slow-growing, locally invasive epidermal tumor with a metastatic rate of <0.1%. Squamous cell carcinomas of the skin and their precursors, actinic keratosis as well as basal cell carcinomas are classified as non-melanocytic skin cancer and belong to the group of epithelial skin tumors. This tumor entity is one of the most common forms of malignant cancer in western countries with an incidence of approximately 100-170 per 100,000 inhabitants per year in Europe.

Basal cell carcinoma accounts for more than 90 percent of all skin cancers in the United States and is the most common of all cancers. Typically, it is a slow-growing cancer that is rarely metastatic, it is an epithelial neoplasm that is believed to derive from the basal layer of the epidermis or follicular epithelium. The classic histologic presentation of BCC is that of nodules and/or strands of atypical basaloid cells that show nuclear palisading, cellular apoptosis, and scattered mitotic activity.

Squamous cell carcinoma (SCC) accounts for the majority of the remainder of cases. Squamous cell carcinoma, also known as squamous cell cancer, is one of the main types of skin cancer that begins from squamous cells in the skin.

SCC and BCC are generally treated by surgical excision, Mohs surgery or electrodessication and curettage. Non-surgical options for the treatment of SCC and BCC include topical chemotherapy, topical immune response modifiers, photodynamic therapy (PDT), radiotherapy, and systemic chemotherapy. The use of topical therapy, such as Imiquimod cream and PDT is generally limited to premalignant (i.e., actinic keratoses) and in situ lesions. Radiation therapy is a primary treatment option for patients in whom surgery is not feasible and is an adjuvant therapy for those with metastatic or high-risk cutaneous SCC. At this time, systemic chemotherapy is used exclusively for patients with metastatic disease. All squamous cell carcinoma lesions are thought to begin via the repeated, uncontrolled division of cancer stem cells of epithelial lineage or characteristics. In contrast to BCC, SCC has a significant recognized rate of metastasis (0.3-3.7%), the majority of which occur from within a subgroup of high-risk SCC.

Incidence of NMSC has significantly increased up to 10% per annum, and currently 2-3 million new cases of NMSC are diagnosed worldwide every year. Most countries do not have cancer registries for NMSC and reported figures are likely to be underestimated. Incidence rates of NMSC increase closer to the equator, with the highest reported rates in the northern territories of Australia.

Actinic keratosis caused by chronic sun damage to the skin and is considered a milder form of NMSC. Actinic keratosis is usually the first lesion in a disease continuum that progresses to invasive SCC. The risk of progression from actinic keratosis to SCC depends on a number of patient factors. The risk of transformation is enhanced in patients with increased solar damage, immunosuppression, and those of advanced age. The presence of actinic keratosis is a risk factor for non melanoma skin cancers and they are precursors of squamous cell carcinoma. The treatment of actinic keratosis is also very important to prevent the development of the disease in the SCC.

Infection, scarring and hemorrage are significant risks involved with curettage, cautery and other surgical options. Although radiotherapy has been efficient in the treatment of NMSC, it has been shown to increase the risk of subsequent BCC and SCC. Toll-like receptor (TLR) agonists, such as imiquimod, are efficient in the treatment of certain types of NMSC but they induce local skin reactions in a large percentage of the patients.

It is known that oncogenic viral strains can develop into cancer. Although there have been many developments in cancer research and specifically in this area, current therapies have a high incidence of side effects, which creates patient compliance issues and decreases the efficacy of these therapies. Cervical intraepithelial neoplasia (CIN), also known as cervical dysplasia, is the potentially premalignant transformation and abnormal growth (dysplasia) of squamous cells on the surface of the cervix. CIN most commonly occurs on the cervix at the squamo-columnar junction, but can also occur in vaginal walls and vulvar epithelium. All these infections are considered pre-cancerous conditions of the cervix/anogenital region. The major cause of CIN is chronic infection of the cervix with the sexually transmitted human papillomavirus (HPV), especially the high-risk HPV types 16 and 18. Over 200 types of HPV have been identified. About a dozen of these types appear to cause cervical dysplasia and may lead to the development of cancers of the cervix/anogenital region. There are many similarities between adenocarcinoma and SCC of the cervix, and they are treated mostly in the same manner. However, there are also several differences in epidemiology, prognostic factors, patterns of failure after primary treatment, and possibly in response to specific treatments due to the fact that most new treatments are focused on immunomodulation and the responses vary because the lesions cannot be targeted directly.

There have been more developments in agents that exert their therapeutic effects through apoptosis and immunomodulation, but prior research in necrosis inducing or cytolytic agents has met with difficulty due to their cytotoxicity for healthy cells.

### SUMMARY OF THE INVENTION

The present invention provides a pharmaceutical composition for the treatment of pre-cancerous conditions of the cervix/anogenital region and non-melanoma skin cancers (NMSCs) wherein said composition comprises a surfactant.

In one embodiment of the present invention, a composition for the treatment of pre-cancerous conditions of the cervix/anogenital region and non-melanoma skin cancers (NMSCs) is provided, wherein said composition comprises a surfactant, preferably an anionic or amphoteric surfactant or a pharmaceutically acceptable salt or a stereoisomer thereof, and at least one pharmaceutically acceptable excipient.

In one embodiment of the present invention, a composition for the treatment of pre-cancerous conditions of the cervix/anogenital region and non-melanoma skin cancers (NMSCs) is provided wherein said composition comprises a surfactant, preferably an anionic or amphoteric surfactant, or a pharmaceutically acceptable salt or a stereoisomer thereof, wherein the surfactant further comprises a hydrophilic moiety selected from the group consisting of a carbonate, a sulfonate, and/or a sulfate and at least one pharmaceutically acceptable excipient.

### BRIEF DESCRIPTION OF THE FIGURES

FIG. 1 illustrates the effect of an active surfactant mixture on HaCaT cells in accordance with embodiments of the present invention.

FIG. 2 illustrates the effect of an active surfactant mixture on HeLa cells (HPV-18 positive cervical adenocarcinoma cell line) in accordance with embodiments of the present invention.

FIG. 3 shows results from an MTS assay on HeLa cells 24 hours after treatment in accordance with embodiments of the present invention.

FIG. 4 shows results from an MTS assay on SiHa (SCC cell line) cells 24 hours after treatment in accordance with embodiments of the present invention.

FIG. 5 shows results from an MTS assay on HaCat cells 24 hours after treatment in accordance with embodiments of the present invention.

FIGS. 6 and 7 show the effect of tested compounds on HeLa cell death and HaCat cell death, respectively, in accordance with embodiments of the present invention.

FIG. 8 shows cell viability assay (MTS assay) on A431 cells performed 24 h after treatment with active mixture and corresponding placebo concentration.

FIG. 9 shows cell viability assay (MTS assay) on TE 354.T cells performed 24 h after treatment with active mixture and corresponding placebo concentration.

FIG. 10 shows colony formation ability of A-431 cells treated with active mixture and corresponding placebo concentration.

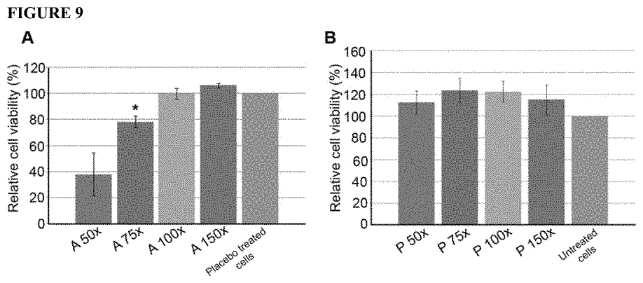

FIG. 11 shows relative colony numbers of A-431 cells treated with active mixture and corresponding placebo concentration.

FIG. 12 shows flow cytometry analysis of Annexin-FITC staining and propidium iodide accumulation after the treatment of A431 cells with the active mixture or placebo.

FIG. 13 shows TE 354.T cells visualization upon drug treatment. A) Untreated cells, B) placebo (50× dilutions), C) Active mix (30× dilutions), D) Active mix (50× dilutions), E) Active mixture (75× dilutions).

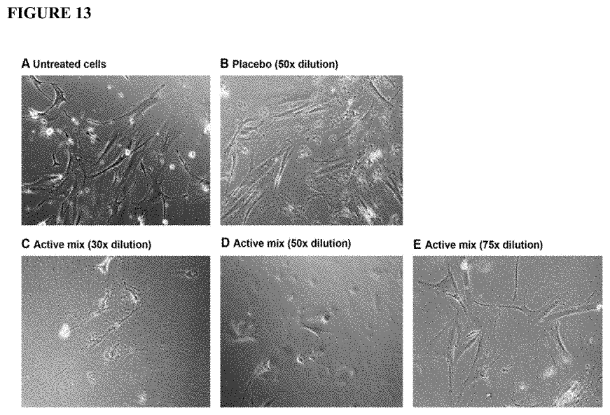

FIG. 14 shows up-regulation of IL-8 by Active mixture in HaCaT cells.

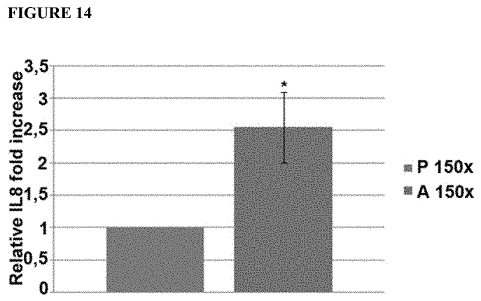

### DETAILED DESCRIPTION OF THE INVENTION

The aim of the present invention is to provide a pharmaceutical composition for the treatment of pre-cancerous conditions of the cervix/anogenital region and non-melanoma skin cancers (NMSCs). It has now been found that surfactants provide the induction of local targeted necrosis through a cytolytic effect therefore effective for the treatment of pre-cancerous conditions of the cervix/anogenital region and non-melanoma skin cancers (NMSCs). The induction of necrosis of the areas affected by SCC (Squamous cell carcinoma), BCC (Basal cell carcinoma), or AK (Actinic Keratosis) through the local application of surfactants has shown to be beneficial in the treatment of these diseases as the surprising effects of the surfactants used on SiHa cells (SCC cell line), TE 354.T (BCC cell line) and A-431 (AK cell line) demonstrate.

Furthermore the same effect also observed on SiHa and HeLa cells (HPV positive cancer cells) in addition to the above mentioned cell lines that also demonstrates the efficacy of the surfactants to help preventing the progression of oncogenic HPV infections to pre-cancerous conditions of the cervix/anogenital region and possibly into cancer.

For the purposes of the present invention a surfactant is used for the treatment of pre-cancerous conditions of the cervix/anogenital region and non-melanoma skin cancers (NMSCs).

In one embodiment of the present invention the surfactant is selected from the group consisting of sodium lauryl sulphate, potassium lauryl sulphate, sodium dodecyl sulphate, potassium dodecyl sulphonate, sodium dodecyl benzene sulphonate, sodium salt of lauryl polyoxyethylene sulphate, lauryl polyethylene oxide sulfonate, dioctyl ester of sodium sulphosuccinic acid or sodium lauryl sulphonate, ammonium lauryl sulfate, sodium 2-ethylhexyl sulfate, sodium octyl sulfate, lithium lauryl sulfate their therapeutically active stereoisomers, their pharmaceutically suitable salt(s), their derivatives and combinations thereof.

In one embodiment of the present invention the surfactant is sodium lauryl sulphate, potassium lauryl sulphate, sodium dodecyl sulphate or potassium dodecyl sulphonate, preferably sodium lauryl sulphate.

In another embodiment of the present invention the surfactant is a molecule shown in Formula (1) below wherein n is an integer from 3 to 25.

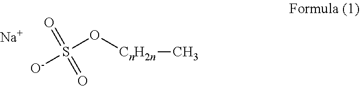

In another embodiment of the present invention the surfactant is selected from the group consisting of the molecules (I) to (LIX) represented by their molecular and structural formulas below, preferably the molecule (LIX).

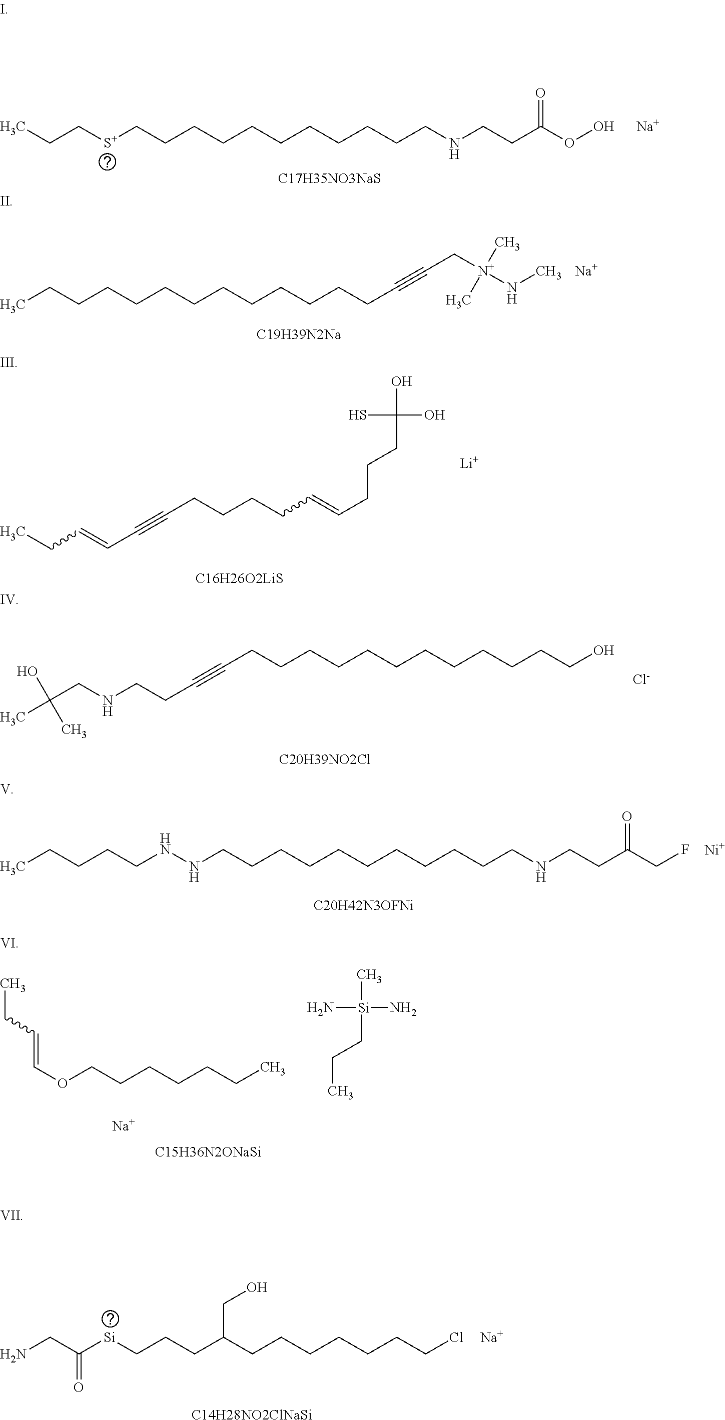

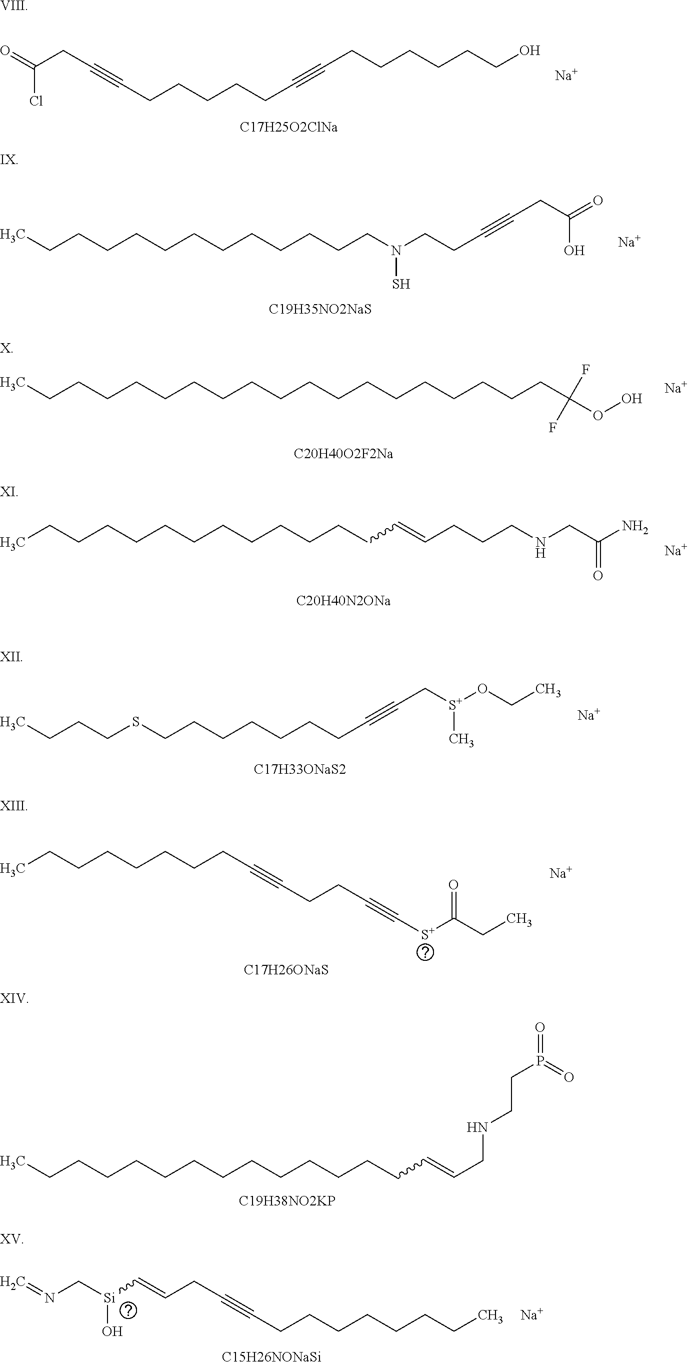

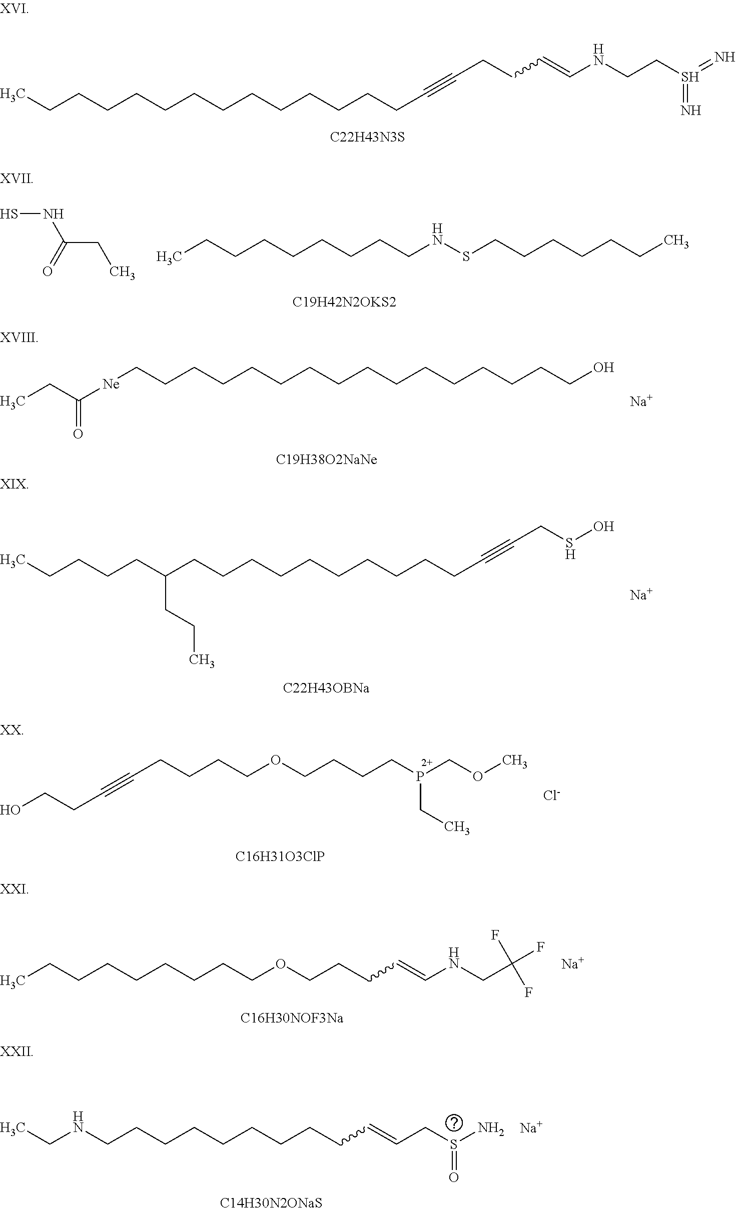

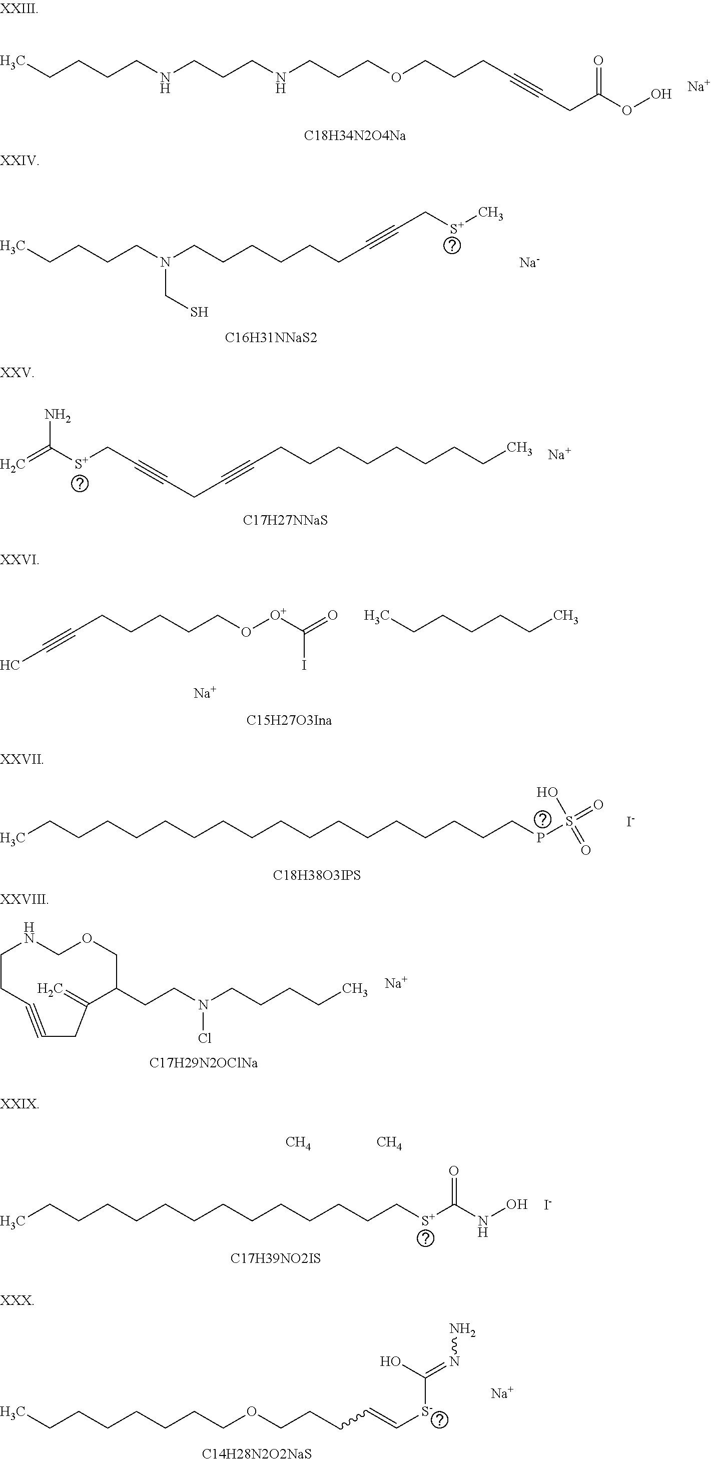

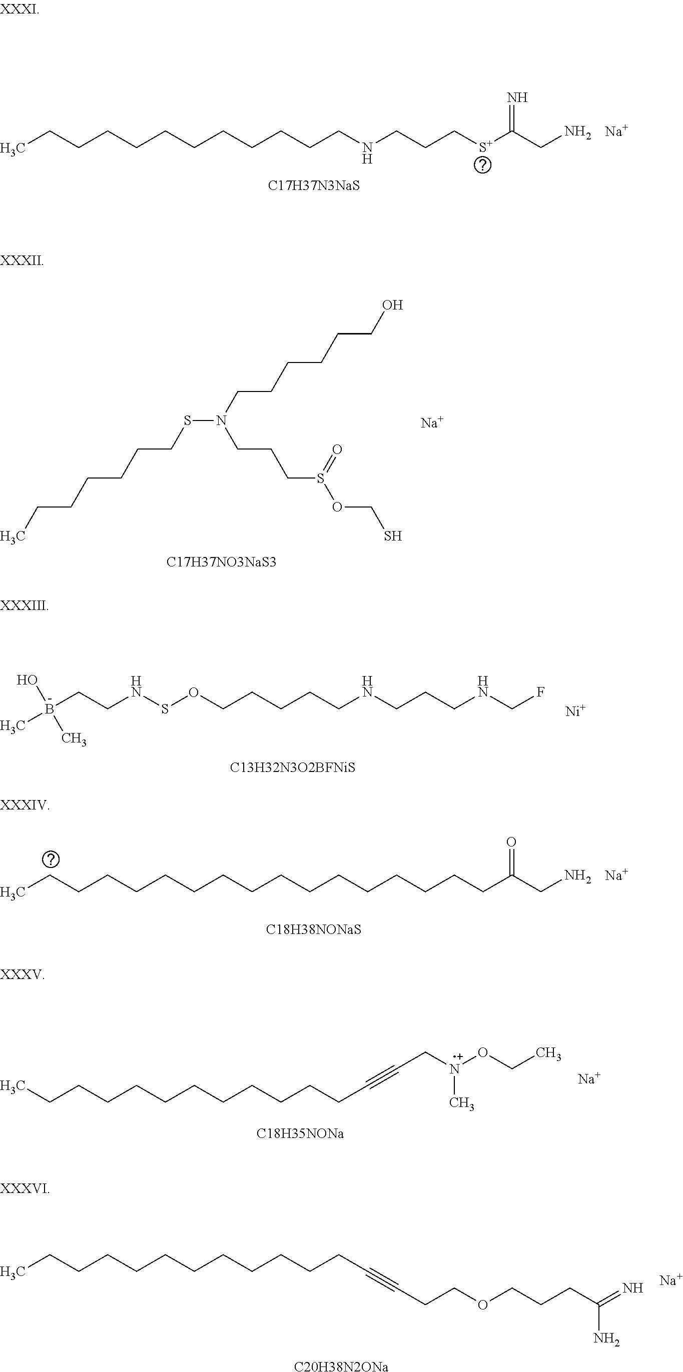

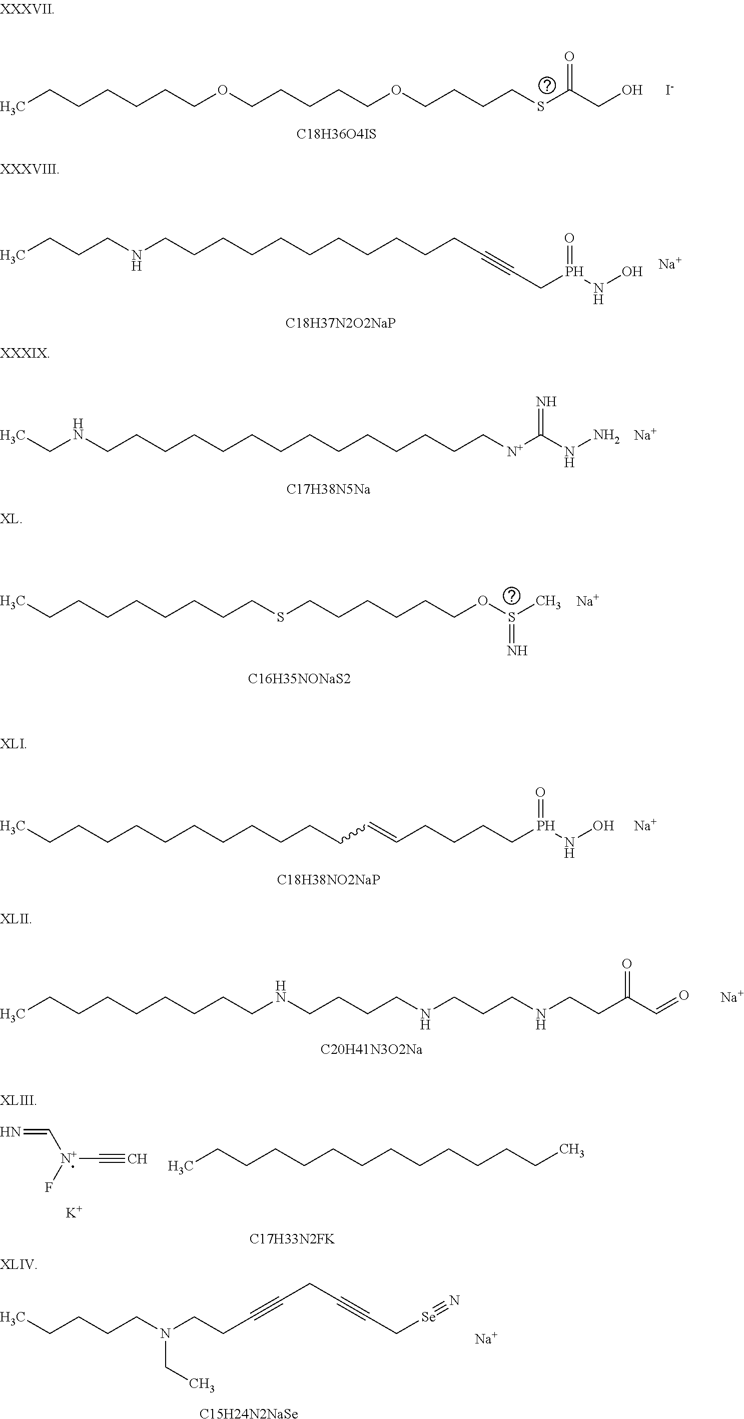

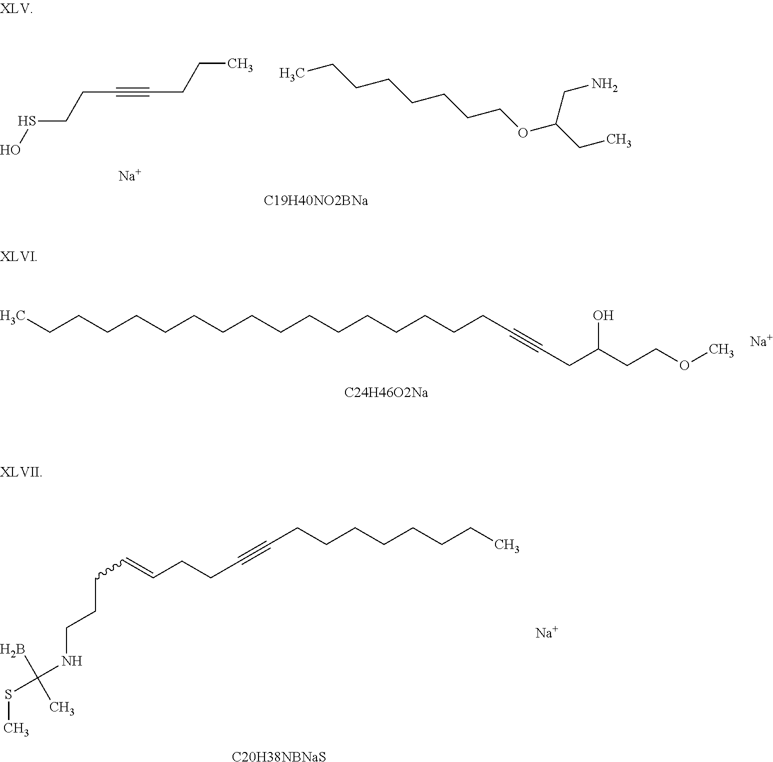

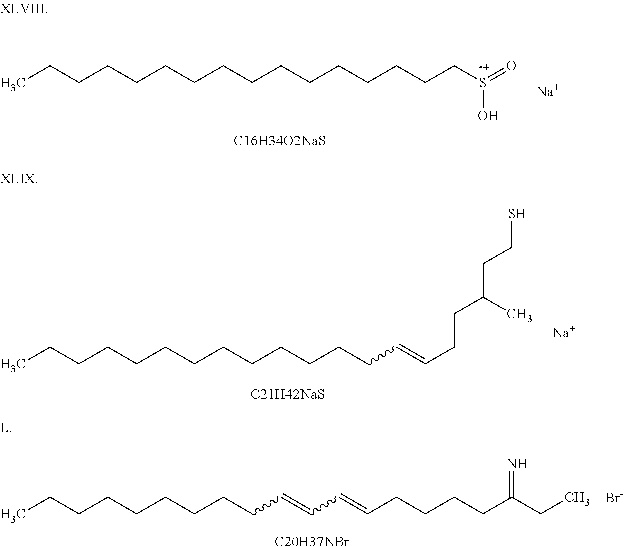

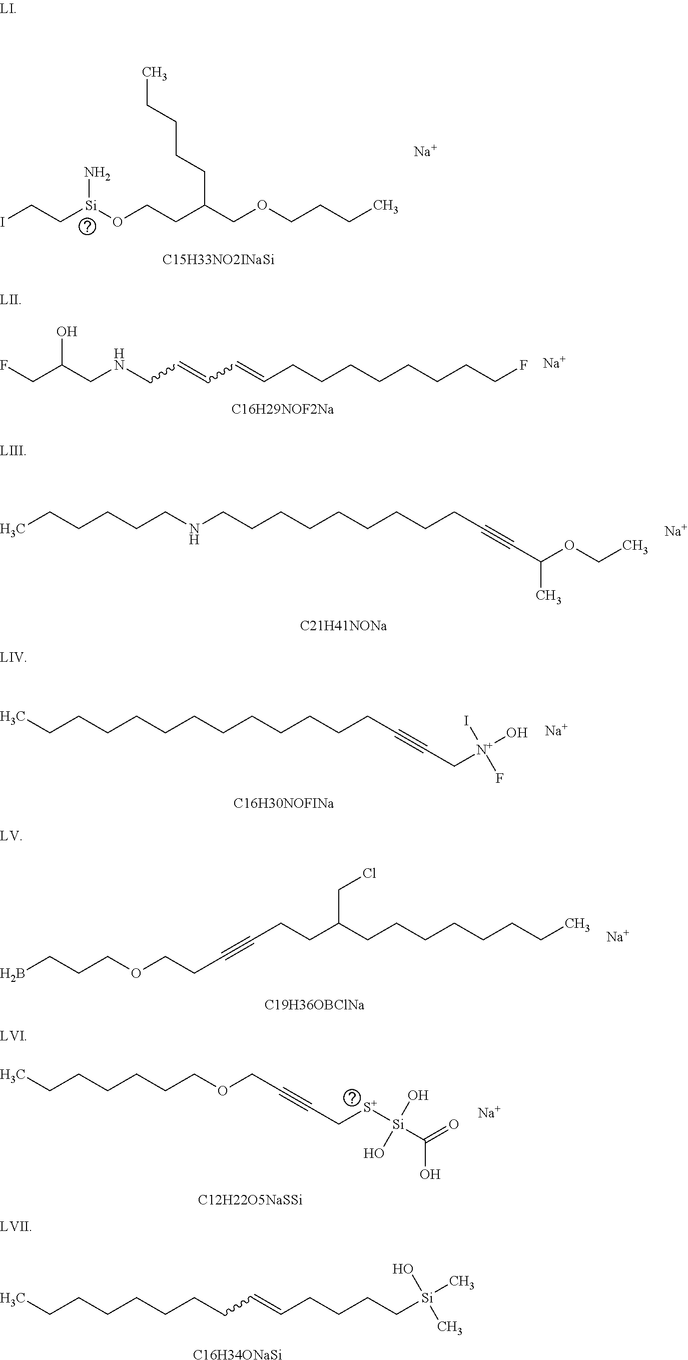

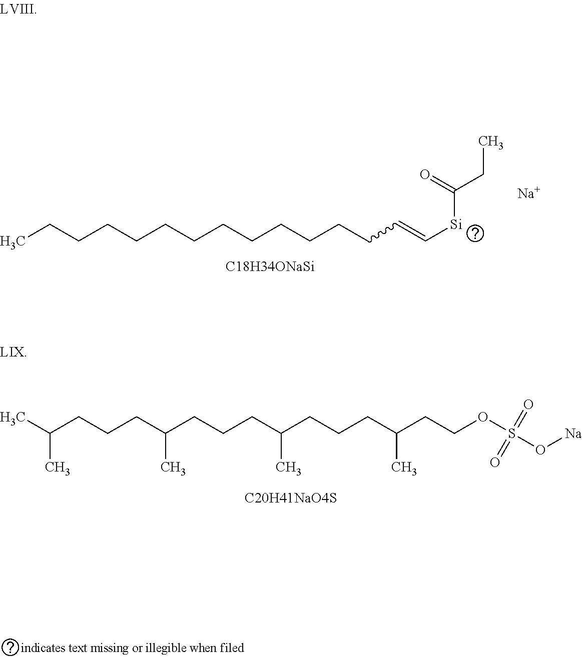

For the purposes of the present invention preferably, anionic and/or amphoteric surfactants are used. Examples of said anionic and/or amphoteric surfactants are sodium lauryl sulphate, potassium lauryl sulphate, sodium dodecyl sulphate, potassium dodecyl sulphonate, sodium dodecyl benzene sulphonate, sodium salt of lauryl polyoxyethylene sulphate, lauryl polyethylene oxide sulfonate, dioctyl ester of sodium sulphosuccinic acid or sodium lauryl sulphonate, ammonium lauryl sulfate, sodium 2-ethylhexyl sulfate, sodium octyl sulfate, lithium lauryl sulfate, cocaminopropionic acid, cociminodipropionic acid, octyliminodipropionic acid, sodium lauriminodipropionate, laurylaminopropionic acid, laurylammobutyric acid, sodium cocaminopropionate, sodium cocaminobutyrate, sodium cociminodipropionate, octadecylaminopropionic acid, sodium octylaminoacetate, and potassium hexadecylaminoacetate, sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethy-7 carboxyamphodiacetate, lauraminopropionic acid, lauroamphodipropionic acid, lauryl aminopropylglycine, lauryl diethylenediaminoglycine,

In one embodiment of the present invention, a composition for the treatment of pre-cancerous conditions of the cervix/anogenital region and non-melanoma skin cancers (NMSCs) is provided, said composition comprising: a surfactant, preferably an anionic or amphoteric surfactant, or a pharmaceutically acceptable salt or a stereoisomer thereof, and at least one pharmaceutically acceptable excipient.

In one embodiment of the present invention, a composition for the treatment of pre-cancerous conditions of the cervix/anogenital region and non-melanoma skin cancers (NMSCs) is provided, wherein said composition comprising one or more surfactants selected from the group consisting of anionic or amphoteric surfactants, preferably anionic surfactants, more preferably sodium lauryl sulphate or derivatives thereof.

In one embodiment of the present invention, a composition for the treatment of pre-cancerous conditions of the cervix/anogenital region and non-melanoma skin cancers (NMSCs) is provided, said composition comprising: a surfactant, preferably an anionic or amphoteric surfactant, or a pharmaceutically acceptable salt or a stereoisomer thereof, wherein the surfactant further comprises a hydrophilic moiety selected from the group consisting of a carbonate, a sulfonate, and/or a sulfate and at least one pharmaceutically acceptable excipient.

In one embodiment of the present invention, a composition for the treatment of pre-cancerous conditions of the cervix/anogenital region and non-melanoma skin cancers (NMSCs) is provided, wherein said composition comprising one or more surfactants selected from the group consisting of sodium lauryl sulphate, potassium lauryl sulphate, sodium dodecyl sulphate, potassium dodecyl sulphonate, sodium dodecyl benzene sulphonate, sodium salt of lauryl polyoxyethylene sulphate, lauryl polyethylene oxide sulfonate, dioctyl ester of sodium sulphosuccinic acid or sodium lauryl sulphonate, ammonium lauryl sulfate, sodium 2-ethylhexyl sulfate, sodium octyl sulfate, lithium lauryl sulfate their therapeutically active stereoisomers, their pharmaceutically suitable salt(s), their derivatives and combinations thereof, preferably sodium lauryl sulphate, potassium lauryl sulphate, sodium dodecyl sulphate, potassium dodecyl sulphonate, sodium dodecyl benzene sulphonate, more preferably sodium lauryl sulphate or potassium lauryl sulphate and a hydrophilic moiety selected from the group consisting of a carbonate, a sulfonate, and/or a sulfate and at least one pharmaceutically acceptable excipient.

In one embodiment of the present invention, a composition for the treatment of pre-cancerous conditions of the cervix/anogenital region and non-melanoma skin cancers (NMSCs) is provided, wherein said composition comprising one or more compounds selected from the group consisting of sodium lauryl sulphate, potassium lauryl sulphate, sodium dodecyl sulphate, potassium dodecyl sulphonate, sodium dodecyl benzene sulphonate, sodium salt of lauryl polyoxyethylene sulphate, lauryl polyethylene oxide sulfonate, dioctyl ester of sodium sulphosuccinic acid or sodium lauryl sulphonate, ammonium lauryl sulfate, sodium 2-ethylhexyl sulfate, sodium octyl sulfate, lithium lauryl sulfate and preferably sodium lauryl sulphate and/or sodium cocoamphohydroxypropylsulfonate and their salts or derivatives thereof.

As used herein, unless otherwise specified, the term “pharmaceutically suitable salt(s)” includes, but not limited to, salts of acidic or basic moieties. Basic moieties are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions. Suitable organic acids include, but are not limited to, maleic, fumaric, benzoic, ascorbic, succinic, acetic, formic, oxalic, propionic, tartaric, salicylic, citric, gluconic, lactic, mandelic, cinnamic, oleic, tannic, aspartic, stearic, palmitic, glycolic, glutamic, gluconic, glucaronic, saccharic, isonicotinic, methanesulfonic, ethanesulfonic, p-toluenesulfonic, benzenesulfonic acids, or pamoic (i.e., 1,1′-methylene-bis-(2-hydroxy-3-naphthoate) acids. Suitable inorganic acids include, but not limited to, hydrochloric, hydrobromic, hydroiodic, sulfuric, phosphoric, or nitric acids.

Compounds that include an amine moiety can form pharmaceutically acceptable salts with various amino acids, in addition to the acids mentioned above. Chemical moieties that are acidic in nature are capable of forming base salts with various pharmacologically acceptable cations. Examples of such salts include, but not limited to; alkali metal or alkaline earth metal salts and, particularly, calcium, magnesium, sodium, lithium, zinc, potassium, or iron salts.

As used herein to describe a compound or chemical moiety, the term “derivative” means a compound or chemical moiety wherein the degree of saturation of at least one bond has been changed (e.g., a single bond has been changed to a double or triple bond) or wherein at least one hydrogen atom is replaced with a different atom or a chemical moiety. Examples of different atoms and chemical moieties include, but not limited to, halogen, oxygen, nitrogen, sulfur, hydroxy, methoxy, alkyl, amine, amide, ketone, and aldehyde.

The surfactant may have one of the following weight percentages in the composition: between 0.1% and 30% by weight of the composition; preferably between 0.3% and 20% by weight of the composition; more preferably between 0.3% and 5% by weight of the composition.

The topical compositions of the invention may be in the form of a gel, cream, ointment, liquid, suspension, solution, emulsion, foam, patch, aerosol or the like for topical administration. Typically, the composition is administered to the subject by spreading (e.g., a gel, cream, or ointment) or spraying (e.g., a liquid) onto the affected area.

In one embodiment, the composition is in the form of a cream. Typically, the cream compositions of the present invention comprise an anionic or amphoteric surfactant and a polyol compound with one or more of an emulsifying agent, a stiffening agent, another surfactant, an emollient, a preservative, an alkalizing or buffering agent, an antioxidant and a solvent.

In one embodiment, the composition is in the form of gel. Typically, the gel compositions of the invention comprise an anionic or amphoteric surfactant and one or more of a rheology modifier or thickener, an alkalizing or buffering agent, and a solvent.

Typically, the composition comprising the anionic or amphoteric surfactant in combination with a polyol compound and/or a cross-linked polyacrylate polymer are administered to the subject in a total daily dose of up to about 500 mg/cm² of skin. The total daily dose may be delivered once per day, or divided between multiple doses. In some embodiments, the composition of the invention may be administered 1, 2, or 3 times per day. In other embodiments, the composition may be administered onto an affected skin area 3 to 14 times weekly by spreading or spraying of the composition onto the affected skin area.

The active agents of the present invention exert their therapeutic effect through cytolysis and the consequential local necrosis of an affected cell population. Although the active agent of the invention includes cytolytic properties, it has been surprisingly found that it has minimal to no side effects to patients. Furthermore it has also been surprisingly found in the studies detailed below, that the active mixture containing a surfactant with anionic properties, for example; SLS (Sodium lauryl sulphate) exerts not only cytolytic but also an immune-modulatory effect as evidenced by the up-regulation of IL-8.

It has also been proven in the study detailed below that surfactants of the present invention do exert an immune stimulatory effect thus increasing the efficiency of cancer treatment and minimizing the risk of recurrence.

Induced cytolysis of affected cell populations, such as those in the cervical or anogenital region infected by oncogenic viral strains, would accelerate release of both mature and immature virus particles that will be exposed to the denaturing potency of the anionic surfactant and will also be subsequently inactivated. A similar surprising effect is evidenced by the use of the surfactant on SiHa cells (bearing SCC) HeLa cells and the BCC and AK cell lines shown in the study below, in which all three cell lines go through necrosis due to the cytolytic effect of the surfactant. Furthermore, massive locally induced cytolytic effect and consequential necrosis due to the cytolytic effect has induced a secretion of proinflammatory cytokines, namely IL-8 which provokes an adaptive immune response that would also limit the recurrence of these conditions.

Pro-inflammatory cytokines have chemo attractant and pro-inflammatory functions. This involves secretion of components that are already present in the cells and released when cells loses integrity of their plasma membrane.

Keratinocytes are able to produce a plethora of cytokines including interleukin (IL)-1, -6, -7, -8, -10, -12, -15, -18, and -20, and tumor necrosis factor α (TNFα), either constitutively or upon induction by various stimuli. This cytokine production by keratinocytes has multiple consequences for the migration of inflammatory cells, may have systemic effects on the immune system, may influence keratinocyte proliferation and differentiation processes, and finally affects the production of other cytokines which can be beneficial as an immune response.

In accordance with embodiments of the present invention, a local targeted treatment, without the burden of systemic administration and a high level of efficacy in the treatment of pre-cancerous conditions of the cervix/anogenital region and non-melanoma skin cancers (NMSCs), creates a new treatment modality for millions of patients suffering from these potentially lethal conditions.

The in-vitro studies detailed below has surprisingly shown that surfactants as detailed above can be a highly effective treatment for the case when targeted necrosis is needed through a cytolytic effect, with mild to no side effects especially when the condition to be treated is a condition involving an epithelial tumor such as BCC and/or SCC (including AK which is a precursor to SCC) or when it is a precancerous condition of the cervix/anogenital region, which are mainly caused by oncogenic viral strains, all of which are evidenced by the surprising discoveries of the studies detailed below and the effects of the of the surfactants of the present invention on HeLa cell line as well. Due to the nature of the cell lines used in the study which are representing both HPV positive SiHa and HeLa cell lines and NMSC cell lines representing BCC and AK and the cytolytic effect of the surfactant inducing necrosis in a dose dependent manner, the present invention provides a completely new treatment modality with minimal side effects through its cytolytic effect, consequential targeted necrosis and immune stimulatory response.

The present invention also has the advantage of its local application which creates a more targeted approach compared with other therapies. This local application creates more exposure of the surfactants to the cancerous or pre-cancerous cells with less side effects.

Another advantage of the present invention is that it does not require a wash off or removal of the applied surfactant, as it has an excellent safety profile as a leave on topical product, even when it was applied daily on the sensitive area of the genital mucosa for more than 8 weeks. Accordingly, the surfactant active ingredients and/or compositions of the present invention may be left on the skin after application and does not require an active removal step.

Furthermore studies below have also surprisingly shown that the treatment with the surfactant composition of the present invention is a better treatment at a shorter treatment period compared to other products on the market, such as aldara (imiquimod 5%) and even compared to imiquimod 3.75% which still has to be removed after 8 hours, whereas the composition of the present invention with a median treatment period of less than 4 weeks does not have to be removed or washed off.

The surprising effect shown and detailed in the in-vitro studies below clearly demonstrate that surfactants and/or compositions of the present invention, and in particular anionic and amphoteric surfactants, have a therapeutic effect for the treatment of diseases which would benefit from the induction of localised targeted necrosis and subsequent adaptive immune response without the usual associated side effects of current therapies in use and in the prior state of the art.

Having demonstrated the excellent safety profile of the surfactants of the present invention and in combination with the surprising results of the in-vitro studies detailed below, it is clearly shown that the present invention is an ideal therapeutic for pre-cancerous conditions of the cervix/anogenital region and non-melanoma skin cancers (NMSCs). Furthermore, the studies have also shown that the active mixtures containing the surfactants of the present invention have more affinity for the cancer and HPV positive HeLa cells, due to the fact that the cytolytic effect was observed earlier in the HPV-18 positive cervical adenocarcinoma cell line (HeLa cells) than in healthy keratinocytes, which helps to explain the excellent safety profile of the present invention, although the present invention is not limited to such a theory in any case.

In-Vitro Study for HPV-18 Positive Cervical Adenocarcinoma Cell Line (e.g. HeLa) and HPV 16 Positive, Squamous Cell Carcinoma Cell Line (e.g. SiHa)

Cells were seeded the day before testing in 12-well plate dishes in complete medium. Testing was performed using the following serial dilutions of active mixture-AM (sodium lauryl sulphate; propylene glycol; carboxypolymethylene) and corresponding dilutions of a placebo (propylene glycol; carboxypolymethylene) as a control:
1. 1× (˜0.8 wt % sodium lauryl sulphate), undiluted AM
2. 2× (0.4 wt % sodium lauryl sulphate)
3. 4× (0.2 wt % sodium lauryl sulphate)
4. 8× (0.1 wt % sodium lauryl sulphate)
5. 10× (0.08 wt % sodium lauryl sulphate)
6. 20× (0.04 wt % sodium lauryl sulphate)
7. 50× (0.016 wt % sodium lauryl sulphate)
8. 100× (0.008 wt % sodium lauryl sulphate)
9. 200× (0.004 wt % sodium lauryl sulphate)

Cells were observed at various time points (immediately upon applying of tested drug, upon 5 min, 1 h and 24 h). High cytolytic effect was observed starting from undiluted substance up to a 20× dilution (lead to complete lysis of the cells immediately after addition or within first hour). Thereafter, decrease in the cytolytic effect was detected. However, it seems that decrease in cytolysis was not completely gradual; at some point the substance was not cytolytic to the cells anymore (around 100× dilution). On the other hand, the placebo did not show a cytotolytic effect in any tested concentration. The overall effect on cell's morphology and survival was similar between healthy keratinocytes (HaCaT), or HPV-18 positive cervical adenocarcinoma cell line (e.g. HeLa). However, in HeLa cells cytolytic effect was observed earlier then in healthy keratinocytes (complete cell loss within first hour vs. 24 hours in the case of HaCaT cells).

During the treatment of HaCat cells with active mixture, cell-cell interactions were lost, membrane integrity was compromised and cell lysis was observed. After 24 hours of treatment, complete lysis was observed. In parallel, the same dilution of a placebo did not result in cell damage.

For HeLa cells, complete cell loss was evident at an earlier time point, compared to HaCaT cells (within first hour) while placebo remained ineffective. Advantageously, the composition of the present invention with an anionic surfactant has more affinity towards HPV bearing oncogenic cells (HeLa cells) rather than healthy keratinocytes (HaCaT cells) which demonstrates why the composition has a very good safety profile although it is cytolytic and induces necrosis.

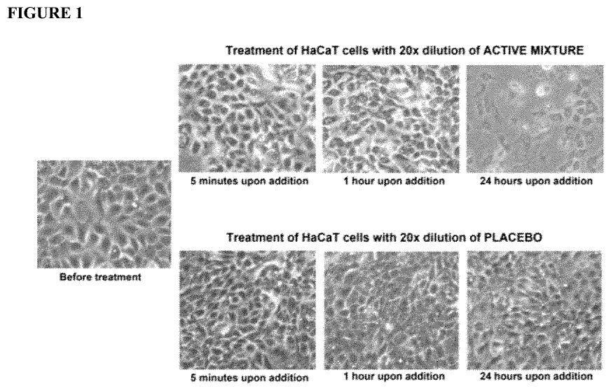

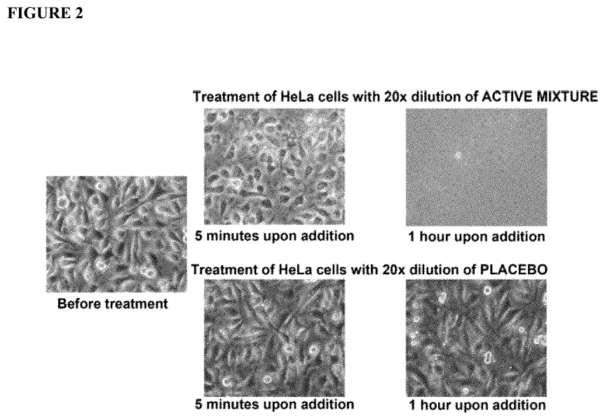

The effect of the tested drug at various concentrations on cell morphology and survival is summarized in Table 1 below, and some representative image captures are presented in FIGS. 1 and 2. FIG. 1 illustrates the effect of an active mixture (20× dilution, ˜0.04 wt % surfactant) and a placebo on HaCaT cells after 5 minutes, 1 hour, and 24 hours. FIG. 2 illustrates the effect of an active mixture (20× dilution, ˜0.04 wt % surfactant) and a placebo on HeLa cells after 5 minutes and 1 hour. As can be seen, cell death occurs over time with the addition of the active mixtures, and at a much higher rate of cell death with the active mixtures as compared to the pacebo.

TABLE 1
The effect of various concentrations of active
mixture on HaCat cell's morphology and survival

| Dilution | Duration of treatment | | | |
|---|---|---|---|---|
| of active | 0 minutes | 5 minutes | 1 hour | 24 hours |
| mixture | Overall effect on HaCaT cell's morphology and/or survival | | | |
| 1x | Complete cell death | | | |
| 2x | Complete cell death | | | |
| 4x | Visible lysis, empty | Complete cell | | |
| | cells | death | | |
| 8x | Lost of cell-cell | Visible lysis | Complete cell | |
| | contact, membrane | | death | |
| | integrity is | | | |
| | compromised | | | |
| 10x | Lost of cell-cell | Visible lysis | Complete cell | |
| | contact, membrane | | death | |
| | integrity is | | | |
| | compromised | | | |
| 20x | Majority of cells look | Lost of cell-cell | Cell leakage is | Complete cell |
| | viable | contact, membrane | visible, cells | death |
| | | integrity is | appear empty | |
| | | compromised in | | |
| | | many cells | | |
| 50x | Majority of cells look | Majority of cells | Some cell-cell | Cell leakage is |
| | viable | look viable | contacts are | visible, some |
| | | | lost, many | cells are still |
| | | | viable cells | unharmed |
| | | | remained | |
| 100x | Viable cells | Viable cells | Viable cells | Majority of |
| | | | | viable cells |
| 200x | Viable cells | Viable cells | Viable cells | Majority of |
| | | | | viable cells |

MTS Assays (Cell Viability Test)

Since dilutions ranging from 1× to 20× had a high cytolytic effect, for cell viability assay (MTS assay), the following dilutions of active mixture were applied:
1. 50×(0.016 wt % sodium lauryl sulphate)
2. 75×(0.012 wt % sodium lauryl sulphate)
3. 100×(0.008 wt % sodium lauryl sulphate)
4. 150×(0.006 wt % sodium lauryl sulphate)
5. 200×(0.004 wt % sodium lauryl sulphate)

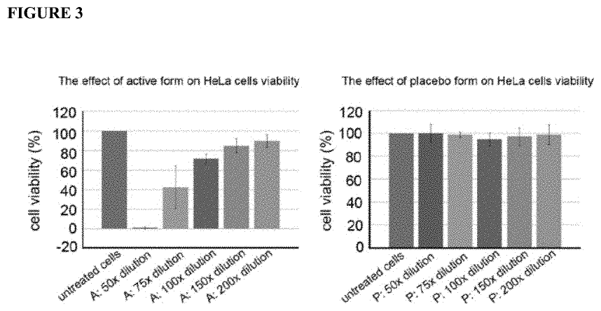

Cells were seeded in 96 well plates the night before treatment and treated with various dilutions of active mixture or placebo for 24 hours. The effect of these treatments was monitored on cell's viability using MTS Cell Proliferation Assay (PromegaCellTiter 96® AQueous One Solution Cell Proliferation Assay). Obtained results are presented in FIGS. 3, 4 and 5.

Referring to FIG. 3, results from an MTS assay on HeLa cells 24 hours after treatment are shown. A: Active mixture; P: Placebo. Relative cell's viability of treated cells was calculated as a percentage of untreated cells viability, which was set as 100%. Data are presented as the mean±standard deviation of at least three independent experiments performed in 6-plicates for each concentration. It was found that the half maximal inhibitory concentration (IC₅₀) for HeLa cells is approximately 90× dilution of active mixture. No effect on cells' viability was observed for the placebo, which shows the surfactant as an active component of the drug.

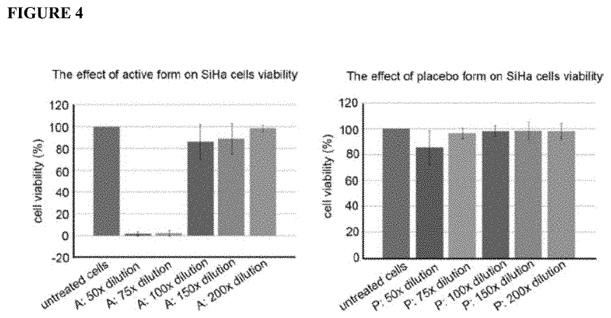

Referring to FIG. 4, results from an MTS assay on SiHa cells 24 hours after treatment are shown. A: Active mixture; P: Placebo. Relative cell's viability of treated cells was calculated as a percentage of untreated cells viability, which was set as 100%. Data are presented as the mean±standard deviation of at least three independent experiments performed in 6-plicates for each concentration. It was found that the half maximal inhibitory concentration (IC₅₀) for was difficult to determine because there is a sharp transition between the effect of 75× dilution (almost complete cytolytic effect upon 24 hours) and 100× dilution (more than 80% of cells survived). This “all or nothing” effect has been observed in all analyzed cell lines (perhaps in a milder form for HeLa cells). As shown for HeLa cells, the placebo composition did not exert any cytotolytic effect.

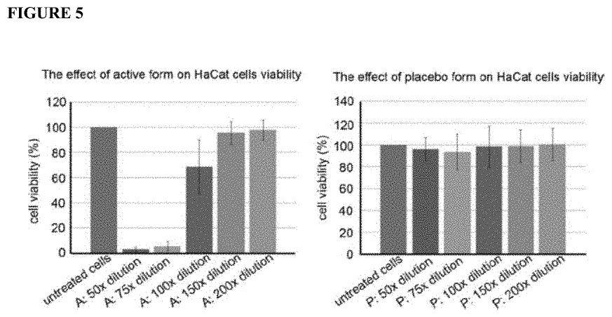

Referring to FIG. 5, results from an MTS assay on HaCat cells 24 hours after treatment are shown. A: Active mixture; P: Placebo. Relative cell's viability of treated cells was calculated as a percentage of untreated cells viability that was set as 100%. Data are presented as the mean±standard deviation of at least three independent experiments performed in 6-plicates for each concentration. It has been found that the half maximal inhibitory concentration (IC₅₀) for HaCat cells is approximately between 80-90× dilution (although “all or nothing” effect was obvious in this cell line as well). No effect on cells' viability was observed for the placebo composition, confirming that the surfactant is an active component of the analyzed mixture.

Annexine V/Propidium Iodide Assay

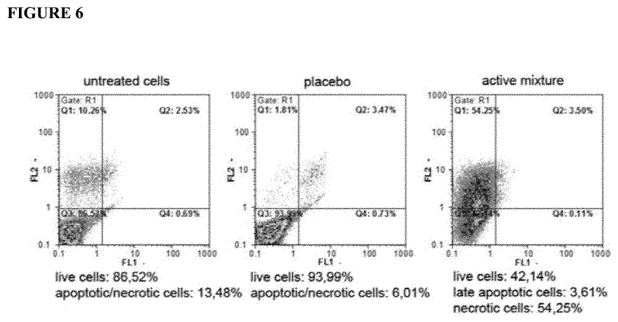

In order to test the effect of surfactants on induction on cell death (apoptosis/necrosis processes) an Annexine V/Propidium Iodide assay was used. Preliminary results on HeLa and HaCat cells (n=1) suggest that the mechanism of cell death is necrosis which is detected by measuring the intensity of red florescent cells stained with Propidium Iodide (PI) by Flow cytometry. By treatment of HeLa cells with 80× dilution of active form of the drug, the integrity of the plasma and nuclear membranes were disturbed, which allowed PI to pass through the membranes and stain the cells. The effect of tested compounds on HeLa cell death and HaCat cell death are illustrated in FIGS. 6 and 7. Preliminary results of the applied apoptosis/necrosis test indicate that the mechanism of cell death upon treatment with an active mixture is cell necrosis. Placebo treatment did not induce the cell death.

In-Vitro Study for Cell Lines Representing BCC and AK.

The aim of the study was to test the effect of SLS, an anionic surfactant on basic cellular processes using non melanoma skin cancer cells representing BCC and AK. The potential of tested formulation to affect cell's viability and proliferation demonstrated its effect on these types of cancer cells. The next aim was to analyze the process leading to cell death induced by active ingredient SLS. Furthermore, the analysis also included the release of pro-inflammatory cytokines upon treatment of healthy keratinocytes with SLS. This was to demonstrate whether SLS was capable to induce immune response in healthy surrounding tissue that could be beneficial for healing process of precancerous or cancerous skin lesions.

Model systems used in this study were:
TE 354.T (ATCC® CRL-7762™) cell line, representing BCC
A-431 (ATCC® CRL-1SSS™) cell line, representing AK.
HaCaT (AddexBio T0020001) cells, spontaneously immortalized, human keratinocyte cell line that has been widely used for studies of skin biology and differentiation.

Task 1.1. Cell viability test upon treatment with drug placebo and active mix

For cell's viability assay (MTS assay) the following dilutions of active mixture were applied:

1. 50× dilution (0,016% of SLS)

2. 75× dilution (0,01% of SLS)

3. 100× dilution (0,008% of SLS)

4. 150× dilution (0,005% of SLS)

5. 200× dilution (0,004% of SLS)

A431 cells (1.5×104 per well) were seeded in 96 well plates a day before treatment, and then treated with various dilutions of active mixture or placebo for 24 hours. After 24 h, cells were supplied with the media containing dye solution and incubated up to 1 h. The effect of these treatments were monitored on cell's viability using MTS Cell Proliferation Assay (PromegaCellTiter 96® A Queous One Solution Cell Proliferation Assay) and colorimetric quantification was done using plate reader (Plate Reader Infinite 200 pro, Tecan).

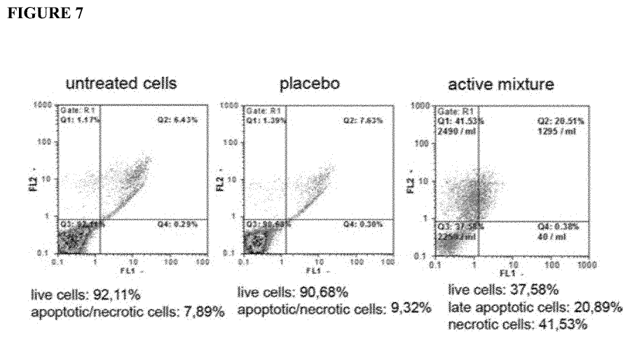

Viability of A431 cells was compromised by the treatment with the different dilutions of active mixture (FIG. 8A). The effect of various dilutions of placebo that correspond to different dilutions of active mixture was tested on cell's viability as well. As presented, tested dilutions of placebo did not affect cell's viability compared to untreated cells (FIG. 8B).

TE 354.T cells (5×103 per well) were seeded in 96 well plates a several days before treatment, and then treated with various dilutions of active mixture or placebo for 24 hours.

As for A431 cells, upon 24 h of treatment TE 354.T cells were supplied with the media containing dye solution and incubated up to 1 h. The effect of these treatments was monitored on cell's viability using MTS Cell Proliferation Assay (PromegaCellTiter 96® AQueous One Solution Cell Proliferation Assay) and colorimetric quantification was done using plate reader (Plate Reader Infinite 200 pro, Tecan). The obtained results are presented in FIG. 2.

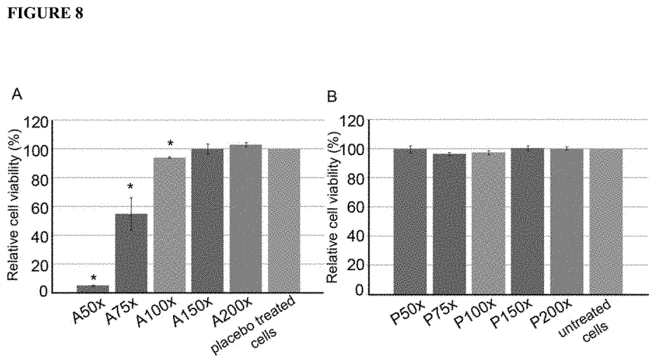

According to data obtained; viability of TE 354.T cells was compromised by the treatment with the different dilutions of active mixture (FIG. 9). The effect of various dilutions of placebo that correspond to different dilutions of active mixture was tested on cell's viability as well. As presented, none of tested dilutions of placebo affected cell's viability compared to untreated cells.

Clonogenic Assay.

Clonogenic assay was also employed to determine the impact of SLS on capability of an A-431 single cell to grow into a colony. The colony is defined to consist of at least 50 cells. The assay essentially tests every cell in the population for its ability to undergo “unlimited” division. This was especially important to demonstrate the capability of a surfactant to halt disease progression.

Visual inspection revealed that the colony formation rate of A-431 cells treated with different dilutions of active mixture was significantly lower than of A-431 cells treated with corresponding dilutions of placebo (FIG. 3A). The number of colonies within the wells of 12-well plates after 10-days treatment of cells with different concentration of active mixture and placebo was counted manually. Obtained results revealed that all applied dilutions of active mixture significantly decreased the colony numbers of the A-431 cells compared to the colony numbers of A-431 cells treated with corresponding dilutions of placebo (FIG. 3B).

10A) Representative images of crystal violet-stained colonies after 10-days treatment with different concentrations of active mixture and placebo. 10B) Relative colony numbers of cells treated with active mixture was calculated as a percentage of colony numbers of cells treated with corresponding dilutions of placebo that was set as 100%. Data are presented as the means±SD (standard deviation) of six independently repeated experiments performed in triplicates for each concentration. Mean values were compared with Student's t-test. The value of p<0.05 is represented by *. Legend—each colour corresponds to a bar presented on histogram; A: —represents different dilutions of active mixture; P: —represents different dilutions of placebo.

The surprising results obtained from the clononegic assay clearly demonstrated the capability of surfactants to halt disease progression.

Cell Death Analysis

In order to test the effect of SLS on induction of cell death (apoptosis/necrosis processes) on non melanoma skin cancer cells Annexin V/Propidium Iodide (PI) assay was used. The Annexin V/PI protocol is a commonly used approach for studying apoptotic cells. PI is a good indicator of cell viability, since it does not stain live or early apoptotic cells due to the presence of an intact plasma membrane. In late apoptotic and necrotic cells, the integrity of the plasma and nuclear membranes decreases, allowing PI to pass through the membranes. Annexin V recognizes phosphatidylserine (PS) in cell membrane. During programmed cell death, soon after initiating apoptosis, PS translocates from the inner face of the plasma membrane to the cell surface. Once on the cell surface, PS can be easily detected by staining with a fluorescent conjugate of Annexin V, a protein that has a high affinity for PS.

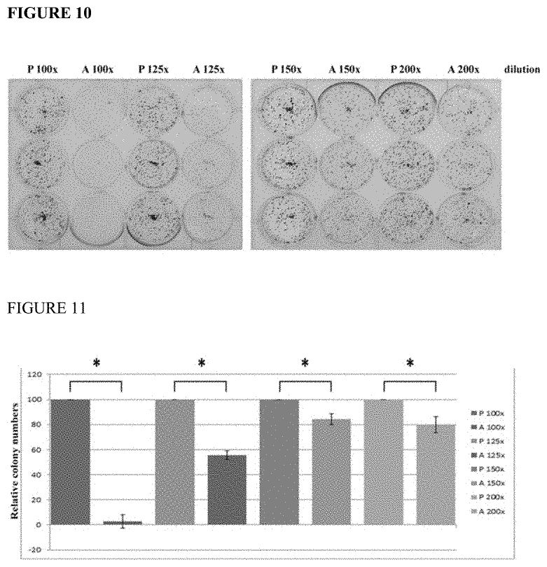

This analysis showed that the necrosis is the main mechanism of cell death induced by active mixture as detected by measuring the intensity of red florescent cells stained with Propidium Iodide (PI) by Flow cytometry (FIG. 11). At the same time the presence of Annexin V positive cells was not observed. By treatment of A431 cell line with IC50 dilution of active mixture the integrity of the plasma and nuclear membranes were disturbed, which allowed PI to pass through the membranes and stain the cells which argues that the mode of action of active mixture studied in vitro is lytic cell death. At the same time, no cell death was observed upon treatment with placebo, which is in correlation with the results obtained for cell viability.

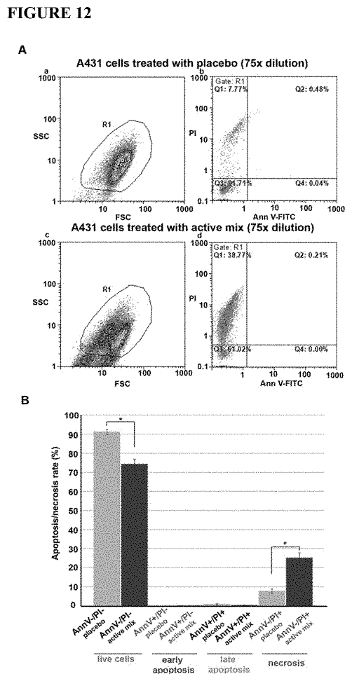

Due to very slow growth capacity of TE 354.T cells, representing BCC, the assessment on flow cytometry was not applicable. Therefore, cells visualization was conducted upon drug treatment (FIG. 12). Visualization showed that cells treated with placebo remained unharmed (FIG. 12B). Upon treatment of cells with 30× dilution of active mixture lower cell number within the optical field was observed, cell-cell interactions were lost and cytoplasm shrinkage was detected (FIG. 12C). This overall reduction in cell volume is one of cell death hallmarks. Treatment with 50× also affected cell number significantly, while 75× dilution had no effect (FIGS. 12E and D).

Detection of pro-inflammatory cytokines released by keratinocytes upon treatment.

In addition to stimulating inflammation, dying cells may also provide signals that help mobilize adaptive immunity (adaptive immune response) to antigens present in the affected tissue. The results of this part of the experiment provides information about possible induction of immune response upon treatment with SLS, which proved the dual role of SLS as a cytolytic agent effective for targeted necrosis as well as an immunomodulator.

ELISA testing showed that the level of IL-8 was increased 2.5-fold upon treatment of HaCaT cells with 150× dilution of Active mixture when compared to the same dilution of Placebo (FIG. 6). In each experiment TNF was used as a positive control for induction of IL-8.

Cells were treated either with 150× dilution of active mixture or placebo for 24 h. Collected supernatants were tested using Human Inflammatory Cytokines Multi-AnalyteELlSArray Kit. Level of IL-8 in supernatants from HaCaT cells treated with 150× Active mixture is expressed as fold increase compared to 150×Placebo-treated cells. Data are presented as means±S.E.M. of five independent experiments.

Induction of cell death by SLS, as an active component of tested drug, induced secretion of IL8 by healthy keratinocytes; production and release of pro-inflammatory cytokines that have an effect on immune response.

The surprising results of the study demonstrate that the active mixture including the surfactants of the present invention can be used for the treatment of non-melanoma skin cancers, conditions caused by oncogenic strains of HPV such as cervical dysplasia, and the like when the lesions/affected area can be locally accessed without any surgical intervention and the composition of the present invention can be directly/locally administered.

For the purposes of the present invention and the claims appended herein, the term “direct or local” means a skin application, including topical application, when the lesions/affected area can be locally accessed without any surgical intervention preferably with a substantially local effect, and a possible minimal systemic effect.

The concentration for the local application of a surfactant composition of the present invention includes 0.2 wt % to 35 wt % of a surfactant. The surfactant is preferably an anionic or an amphoteric surfactant in one example.

Because vaccines would only have effects towards certain oncogenic strains, these would not create a universal prophylaxis for cervical dysplasia or other conditions caused by pre-cancerous cells in the cervical/anogenital region. Advantageously, due to the specific mechanism of action of the treatment of the present invention through targeted necrosis induced through cytolysis, the treatment would not have the limitations and side effects associated with current therapies, systemic administration and will not be only effective against specific oncogenic strains even compared with the most advanced form of vaccines like Gardasil9.

Certain oncogenic HPV strains will not manifest on the skin of the patient as warts but nonetheless can be cancer causing strains (HPV sub types). These strains can be easily diagnosed by a pap smear, even if they are not diagnosable through a visual inspection. In such cases, the administration of a surfactant or surfactants of the present invention would still be an effective treatment in order to prevent these oncogenic strains from turning into cervical dysplasia and furthermore into cervical cancer.

The foregoing embodiments and examples have been provided merely for the purpose of explanation and are in no way to be construed as limiting of the present invention. While the present invention has been disclosed with reference to embodiments, the words used herein are intended to be words of description and illustration, rather than words of limitation. While the present invention has been described with reference to particular materials and embodiments, the present invention is not intended to be limited to the particulars disclosed herein. Rather, the present invention extends to all functionally equivalent structures, materials, and uses, such as are within the scope of the appended claims. Changes may be made, within the purview of the appended claims, as presently stated and as may be amended, without departing from the scope and spirit of the present invention. All terms used in this disclosure should be interpreted in the broadest possible manner consistent with the context.

Female Patient Study for HPV

The new methods and topical compositions for treatment of viral skin infections and their manifestations post infection are based on a randomized double-blind clinical trial in patients with manifestations of anogenital warts (caused by HPV) determined by visual assessment and PCR assays. The results of the study not only show the effectiveness of the surfactans in the treatment of HPV but also that the topical administration of the compositions of the present invention causes minimal to no side effects, such as skin irritations and lesions, which are potential and concerning side effects of existing therapies, and which create major patient compliance issues.

Out of the 82 female patients enrolled in the study, 38 were given a topical formulation containing an anionic surfactant, in combination with at least one polyol compound and/or a cross-linked polyacrylate polymer in accordance with embodiments of the present invention in a double-blind randomized manner. All of the participants were female subjects 18 years and older, all of w were diagnosed with anogenital condylomata acuminata (HPV).

During the study patients were assessed and questioned by the investigator to determine the efficacy and possible side effects of the formulation. 32 patients out of 38 patients had complete clearance using the surfactant composition of the present invention, 2 patients did not have complete clearance, and 4 patients dropped out of the study for reasons unrelated to the topical formulation. Thus, 32 out of 34 patients that adhered to the use of the formulation had complete clearence for a greater than 90% complete clearance rate. More than 50% of the patients using the formulation that can be devised according to the present invention, had complete clearance of genital warts within the first 2 weeks. 20% of patients had complete clearance within the first 4 weeks, and another 20% had complete clearance within the first 6 weeks.

The study has provided evidence of unparalleled clinical effect of the topical composition of the present invention. The study led to the discovery that the topical administration of an anionic or amphoteric surfactant in combination with at least one polyol compound and/or a cross-linked polyacrylate polymer had a positive effect on more than 90% of the study participants and treated the conditions and manifestations associated with genital HPV with minimal side effects. These viral infections and their manifestations are not adequately treated with existing drugs including interferons, immiquimod, and DNA sythesis inhibitors such as nucleoside, purine and prymidine analogues.

In comparison, the study with the topical formulation of the present invention has demonstrated a complete clearence rate with more than 90% of patients, without recurrence in a maximum time period of 6 weeks, with most of the patients being completely manifestation-free within the first 2 weeks. This is unprecedented in treating the manifestations of HPV, especially considering that only minimal side effects, such as a minor burning sensation occured and these were not significant enough for the patients to drop out of the study.

### EXAMPLES

### Example 1: The Preparation Method of One of the Surfactants According to the Present Invention

### Synthesis of C20H41NaO4S

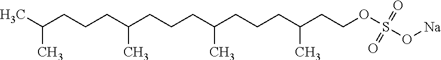 [0138] Phytol (25.0 g, 84.3 mmol) was hydrogenated at 25° C. in ethanol (250 mL) at 1013 mbar for approx. 6 h.
The reaction mixture was passed through a pad of celite to remove the catalyst and the resulting filtrate was concentrated in vacuo
Purification on silica gel resulted in 17.27 g (57.8 mmol) of 3,7,11,15-Tetramethyl-hexadecan-1-ol (17.27 g, purity: >95% by GC, yield: 68%).
Under inert atmosphere, alcohol (10.18 g, 34.1 mmol) was dissolved in CH2Cl2 (10 mL) and cooled to −5° C. Chlorsulfonic acid (2.50 mL, 37.5 mmol, 1.1 equiv) was added dropwise thereto.
After addition was complete, the reaction mixture was stirred under cooling for approx. 2 h and then allowed to reach 23° C. within 1/2 h.
All volatiles were evaporated in vacuo providing a red-brown oily residue which was dissolved in methanol (20 mL) and cooled to +5° C.
A solution of sodium methoxide (30% in methanol, ˜10 mL) was added thereto until the pH of the reaction mixtures was about 9-10. Upon neutralisation, the product precipitated. After isolation, it was re-dissolved and purified by flash chromatography. And the final product with the molecular formula: C20H41NaO4S was obtained (5.17 g, purity: >90% by NMR, yield: 38%).

### Example 2: Cream Formulation

Table 2 below provides the contents of an example composition in the form of a cream.

TABLE 2
Cream Composition

| Illustrative Cream Composition | Amount |
|---|---|
| Ingredients | (% by weight of the composition) |
| Stiffening agent | about 1%-45% |
| Cross-linked polyacrylate polymers | about 0.1%-50% |
| Anionic or amphoteric surfactant | about 0.1%-50% |
| Preservative | about 0.01%-0.6% |
| Polyol compound | about 0.1%-50% |
| Alkalizing or buffering agent | about 0.01%-3% |
| Antioxidant | about 1%-15% |
| Emollient | about 1%-50% |
| Solvent (e.g., distilled water) | q.s. (e.g., 20%-80%) |

### Example 3: Gel Formulation

Table 3 below provides the contents of an example composition in the form of a gel.

TABLE 3
Gel Composition

| Illustrative Gel Composition | Amount |
|---|---|
| Ingredients | (% by weight of the composition) |
| Anionic or amphoteric surfactant | about 0.1%-50% |
| Polyol compound | about 0.1%-50% |
| Cross-linked polyacrylate polymer | about 0.1%-50% |
| Rheology modifier or thickener | about 0.5%-2% |
| Alkalizing or buffering agent | about 0.5%-10% |
| Solvent (e.g., distilled water) | q.s. (e.g., 20%-80%) |

1.-18. (canceled)

19. A method of treating pre-cancerous conditions of the cervix/anogenital region or non-melanoma skin cancers (NMSCs), the method comprising administering to an individual in need of such treatment a composition comprising a surfactant wherein the surfactant is selected from the group consisting of sodium lauryl sulfate, potassium lauryl sulfate, sodium dodecyl sulfate, potassium dodecyl sulfonate, sodium dodecyl benzene sulfonate, sodium salt of lauryl polyoxyethylene sulfate, lauryl polyethylene oxide sulfonate, dioctyl ester of sodium sulfosuccinic acid, sodium lauryl sulfonate, ammonium lauryl sulfate, sodium 2-ethylhexyl sulfate, sodium octyl sulfate, lithium lauryl sulfate, their salts and derivatives thereof.

20. The method of claim 19 wherein the surfactant is selected from the group consisting of sodium lauryl sulfate, sodium dodecyl sulfate, potassium dodecyl sulfonate, sodium dodecyl benzene sulfonate, lauryl polyoxyethylene sulfate, dioctyl ester of sodium sulfosuccinic acid, sodium lauryl sulfonate, their salts and derivatives thereof.

21. The method of claim 19 wherein the composition is in the form of a topical gel, cream, ointment, liquid, suspension, solution, emulsion, foam, patch or aerosol.

22. The method of claim 19 wherein the composition further comprises one or more of a suitable pharmaceutical excipient, including a preservative, rheology modifier or thickener, an alkalizing or buffering agent, and a solvent.

23. The method of claim 19 wherein the surfactant is in a combination with a polyol compound and/or a cross-linked polyacrylate polymer, and administered to the subject in a total daily dose of up to about 500 mg/cm2 of skin.

24. The method of claim 19 wherein the surfactant is between 0.1% and 30% by weight of the composition.

25. The method of claim 19 wherein the surfactant is between 0.3% and 20% by weight of the composition.

26. The method of claim 19 wherein the surfactant is between 0.3% and 5% by weight of the composition.

27. The method of claim 19 wherein the non-melanoma skin cancer is basal cell carcinoma, squamous cell carcinoma or actinic keratosis.

28. The method of claim 19 wherein the composition is administered to the affected area 1 to 3 times daily.

29. The method of claim 19 wherein the total daily dose is delivered once per day, or divided between multiple doses.

30. The method of claim 19 wherein the surfactant is sodium lauryl sulfate or salts and derivatives thereof.
